(19) United States
(12) Patent Application Publication
(10) Pub. No.: US 2012/0222688 A1
(43) Pub. Date: Sep. 6, 2012

(54) Protective shield for male genitalia

(76) Inventors: Mark Farner, Petoskey, MI (US); Lesia Farner, Petoskey, MI (US)

(21) Appl. No.: 12/932,690

(22) Filed: Mar. 3, 2011

(51) Int. Cl. A61F 13/00

(52) U.S. Cl. 128/889

(57) ABSTRACT

A protective shield for use on a male penis comprises a rectangular shaped member, wherein the member is flexible and removably securable to the penis and itself. The protective shield also comprises a remover mechanism secured to an end of the member. The protective shield also comprises a guideline arranged along at least one edge of the member. The protective shield will be used to wrap the head and a predetermined portion of the shaft of the penis to protect it from abrasion encountered by males in everyday living.

### BACKGROUND OF THE INVENTION

1. Field of the Invention

The present invention generally relates to a protective shield for a male penis, and more particularly, relates to a wrap for use on a male genitalia that will shield the penis from abrasions like the intention of the natural foreskin.

2. Description of Related Art

Sleeves and condoms for use on a male penis have been known for many years in the art. Many men have experienced erectile dysfunction (ED), which may be caused by years of penal abrasion and callousing that is generally only found in circumcised men who use no protection on their penis during every day living. Generally, in the prior art, ED is treated and prevented through prescription medicines or herbs sold over the counter at health food stores and the like. While these prescription drugs and over the counter medications may in fact help increase the ability of a man to get an erection, these drugs and supplements have side effects that may deter some men from seeking a solution to their ED problem. Some of the sleeves or covers disclosed in the prior art for a male penis deal with incontinence, prevention of pregnancy and prevention of the transfer of sexually transmitted diseases. These protective coverings, such as condoms, bags or balloon type devices that collect urine and protect from impregnation of a female or transmission of sexual disease between a male and a female generally are one use devices that do not protect, help to avoid or overcome penile abrasion or callousing generally found on circumcised men.

Therefore, there is a need in the art for a sterile, biodegradable, hypoallergenic, breathable and stretchy protective shield for a male penis. There also is a need in the art for a protective shield that is easy to remove and place on a penis and is comfortable enough to wear for daily use on a male without unwanted discomfort and bulkiness around the male penis. Furthermore, there is a need in the art for a protective shield for the penis that is capable of being reused and reapplied when removed for urination and sexual acts. There also is a need in the art for a protective shield that has a guideline arranged along at least one edge thereof and a remover mechanism secured to an end of the protective shield for use in removing the protective shield from the male penis by the user.

### SUMMARY OF THE INVENTION

One object of the present invention may be to provide a protective shield for a penis.

Another object of the present invention may be to provide a protective shield for a male penis that is reusable and removable.

Still a further object of the present invention may be to provide a method of protecting a male penis via the use of a sterile, biodegradable, hypoallergenic, breathable and stretchy protective shield wrapped around the head and a predetermined portion of the shaft of the penis.

Still another object of the present invention may be to provide a protective shield for a male penis that includes a pull string attached to one end thereof for easy removal of the protective shield.

Still another object of the present invention may be to provide a protective shield for a penis that may include a guideline or finder line to assist in orientation of the protective shield with relation to the penis during application thereof.

Another object of the present invention may be to provide a protective shield for a male penis to increase sensitivity to the skin at the head of the penis.

Still another object of the present invention may be to provide a protective shield for a male penis that will allow the texture of the head of the penis to become smoother and softer through continuous use of the protective shield.

To achieve the foregoing objects, a protective shield for use on a male penis is disclosed. The protective shield comprises a rectangular shaped member or body wherein that member is flexible and removably securable to itself and the penis. The protective shield also comprises a remover mechanism secured to an end of the member. The protective shield also includes a guideline arranged along at least one edge of the member. The guideline will be used to help orientate the protective shield to the penis during application of the shield onto the penis.

One advantage of the present invention may be that it provides for a protective shield for a penis.

A further advantage of the present invention may be that it provides for a protective shield that will wrap around the head and a small portion of the shaft of the penis.

Still another advantage of the present invention may be that it provides for a protective shield that will guard against callousing due to abrasion on the head of the penis and the end of the shaft of the penis much like natural foreskin.

Still another advantage of the present invention may be that the protective shield may allow men to regain some of the loss sensitivity in the head of their penis which may increase their sexual pleasure due to the ability to feel what in the past had been calloused.

Still another advantage of the present invention may be that the protective shield for the male penis may be a way to avoid or overcome erectile dysfunction caused by years of penal abrasion and callousing generally found in circumcised men who use no protection during everyday living.

Still another advantage of the present invention may be a protective shield for the penis that includes a pull string arranged on one end thereof.

Still another advantage of the present invention may be a protective shield for the penis that includes a finder line or guideline arranged on at least one edge of the member of the protective shield to assist in orientation of the shield with relation to the penis during application thereof.

Yet another advantage of the present invention may be a protective shield for a penis which may be used constantly without interfering with every day life.

Yet another advantage of the present invention may be a protective shield for the penis that is reusable and removable by the user.

Other objects, features and advantages of the present invention will become apparent from the subsequent description and the appended claims, taken in conjunction with the accompanying drawings.

### BRIEF DESCRIPTION OF THE DRAWINGS

FIG. 1 shows a protective shield for a penis according to the present invention.

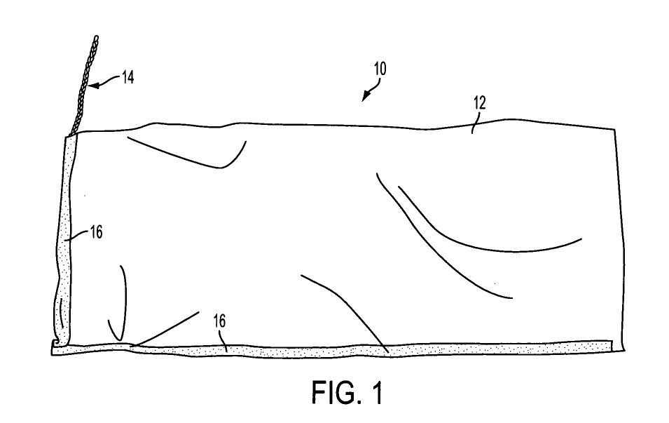

FIG. 2 shows a protective shield at the beginning of application to a penis according to the present invention.

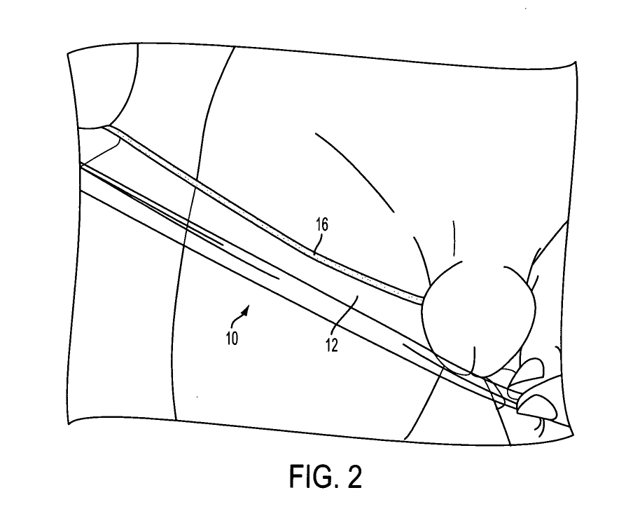

FIG. 3 shows a penis wrapped in the protective shield according to the present invention.

### DESCRIPTION OF THE EMBODIMENT(S)

Referring to the drawings, a protective shield 10 for use on a male penis and a method of applying is shown. Generally, the protective shield 10 may be used on a male penis of a male who has been circumcised, i.e., had foreskin removed from their penis. The protective shield 10 generally may be a sterile, biodegradable, hypoallergenic, breathable and flexible or stretchy device that may allow for protection of the male penis. The protective shield 10 of the present invention may guard against callousing of the circumcised penis that generally occurs due to abrasion during every day living by a male. The protective shield 10 of the present invention may act and provide similar protection to the penis as that intended by the natural foreskin of a male penis. It is contemplated that by continuous of the protective shield men who have been circumcised may regain some of the loss sensitivity in the head of their penis and may also notice an increase in their sexual pleasure because of the ability to actually feel what they could not because their penis had been calloused and desensitized due to abrasion by undergarments and clothing used in every day living. The protective shield 10 of the present invention may in fact provide for a way to avoid or overcome erectile dysfunction caused by penal abrasion and callousing that is generally found in circumcised men who use no protection during their daily living. Furthermore, the use of the protective shield 10 for the penis may be able to replace prescription drugs and herbal supplements that are sold over the counter, which may have undesirable side effects which may persuade some men not to even attempt to use such medications.

Generally, for circumcised men abrasion and callousing of the penis is unavoidable if no protection is provided against a slow and methodical desensitization of the head of a circumcised penis because of the constant rubbing and abrasion of the penis against undergarments and clothing. The use of the protective shield 10 according to the present invention may in fact increase the sensitivity of the head of the male penis. The protective shield 10 may also encourage a metamorphosis that occurs to the skin at the head of the penis. This metamorphosis at the head of the penis allows for the texture of the skin at the head of the penis to become smoother and softer and may in fact produce the natural fermions associated with its original environment as enclosed in the natural foreskin, which has been removed due to circumcision.

As shown in the drawings, the protective shield 10 for the penis generally includes a rectangular shaped member or body 12 having any known dimensions. However, in one contemplated embodiment the protective shield 10 is approximately twelve inches long by four and one half inches wide. The protective shield 10 with its generally rectangular shape may have two long edges and two shorter edges to form the rectangular shape. It should be noted that it is also contemplated that any other dimensions may be used for the protective shield 10 according to the present invention. Furthermore, it should be noted that any other shape, other than a rectangular shape, may be used for the protective shield 10 for the penis according to the present invention including but not limited to a square shape, a triangular shape, a circular shape, an oval shape, any polygonal shape, or any other known shape or random shape may be used for the protective shield body 12 for the penis. In the embodiment shown, the protective shield 10 is made of a cellophane material. However, it is contemplated to use any known polyethylene material, plastic material, natural material, composite material, or any other known material that is capable of being flexible and removably securable to itself and the penis. In the embodiment shown a clear cellophane material is used, however it is contemplated to use any known color for the protective shield member 12 for the penis. The body 12 of the protective shield 10 for the penis may even have multi colors and different designs arranged thereon for unique looks and designs for the shield 10 wrapped around the male penis. It should also be noted that any known thickness of the body 12 of the protective shield 10 may be used according to the present invention.

The protective shield 10 according to the present invention and as shown in the drawings may also include a remover mechanism 14 secured to one end or edge of the body 12 of the protective shield 10. In the embodiment disclosed, the remover mechanism 14 may be secured to one of the shorter edges of the protective shield 10. However, it is also contemplated to attach the remover string 14 to either of the longer edges or to one or both of the shorter edges or one or both of the longer edges. The remover mechanism 14 in one contemplated embodiment will be a pull string that will allow for easy removal of the protective shield 10 after application or installation and wearing on the male penis. Any known mechanical or chemical fastening techniques may be used to secure the remover mechanism or pull string 14 to the end of the protective shield 10 as shown in the invention. It is contemplated to use a glue or other hypoallergenic material to secure the pull string 14 to the end of the protective shield body 12. It is also contemplated to use any known mechanical fastening or any other chemical fastening technique to secure the string to the protective shield body 12. It is also contemplated to manufacture the remover mechanism 14 directly into the protective shield 10 as an integral piece. Furthermore, it is contemplated to add the pull string 14 after manufacture of the protective shield 10 such that it is an add-on portion to the protective shield 10. It should be noted that any type of material may be used for the remover mechanism 14 including but not limited to natural materials, plastic materials, composite materials, or any other known material capable of being formed into a general cylindrical shape or any other known shape may also be used for the remover mechanism 14. The remover mechanism 14 may extend the entire length of one of the short edges of the protective shield 10 or may only be secured to a predetermined portion of the shorter edge of the protective shield 10. It should further be noted that the remover mechanism 14 may extend a predetermined distance from the edge of the protective shield 10 such that removal of the protective shield 10 is easy for the user. The remover mechanism 14 may extend any distance from the edge of the protective shield 10 to allow for customization of the protective shield 10 by the user.

The protective shield 10 may also include a guideline or finder line 16 that may assist in orientation of the protective shield 10 to the male penis during installation and application of the protective shield 10 over the male penis. In one contemplated embodiment, the guideline 16 may be arranged along one of the long edges of the protective shield 10 and one of the short edges of the protective shield 10. In this embodiment when viewing the protective shield 10 from above, the guideline or finder line 16 may form an L-shape. It is also contemplated to use the guideline 16 around the entire outer periphery of the protective shield 10 or on one edge of the protective shield 10 or on one predetermined portion of one edge of the protective shield 10 or on multiple edges of the protective shield 10. It should be noted that the guideline 16 may be made with raised members to allow for use by people with limited sight or may, as shown in the present embodiment, be a different color to assist those with ample sight in correctly orientating the protective shield 10 prior to application or putting of the protective shield 10 around the male penis. It should be noted that in one contemplated embodiment the finder line 16 may be a different color that that of the body 12 of the protective shield 10. This will allow for the finder line 16 to be easily seen and orientated with relation to the male penis.

It is also contemplated in another embodiment that the protective shield body 12 on the leading edge of the protective shield 10, i.e., the part that covers the penis head, may have a predetermined portion of this leading edge frosted or coated with a predetermined material or by a predetermined process. This predetermined material or process may prevent the protective shield 10 from sticking to itself, thus providing an air channel much like the natural foreskin of a penis, thus may be reducing the risk of a rash or infection setting in on the penis. Furthermore, the use of this frosted region may oxygenate the penis head also like the natural foreskin does. In one contemplated embodiment this frosted or coated portion of the leading edge of the protective shield 10 may be a strip of approximately one inch in width along the entire leading edge of the body 12 of the protective shield 10. As noted above, the frosting of this predetermined length strip at the leading edge of the body 12 of the protective shield 10 may be done during the manufacturing process in the form of physical treatment which will allow for the end of the protective wrap 10 from cutting off the opening thus allowing for ample air exposure and flow to the penis head. Any known frosting or coating methodology or technology may be used to treat or affix this predetermined width strip along the entire leading edge of the protective shield 10. It should be noted that the width of the frosted portion of the protective shield 10 may be from an eighth of an inch to three inches depending on the size of the protective shield 10 and the use therefore.

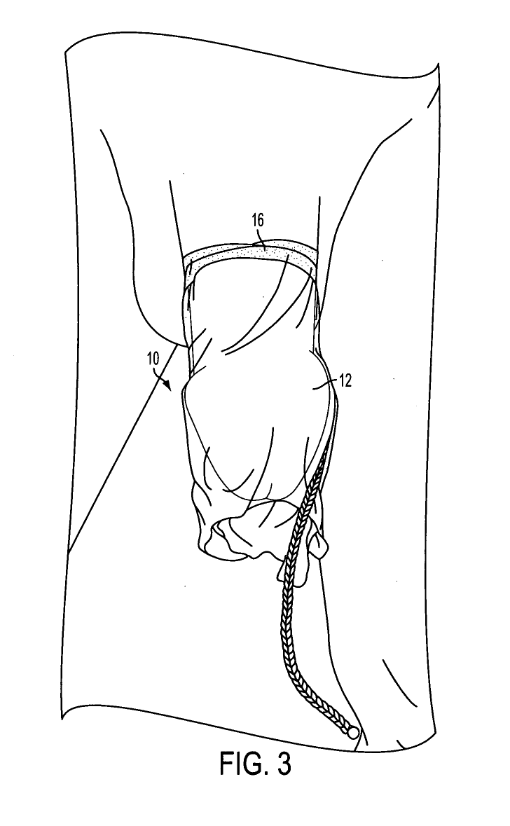

During operation, the male may align one short edge of the protective shield 10 along a longitudinal line or axis of the penis. The protective shield 10 is arranged such that it covers a predetermined portion of the shaft of the penis and completely covers the head of the penis. After aligning the protective shield 10, with assistance from the finder line 16, in a longitudinal manner with relation to the penis, the user will wrap or rotate the opposite or free end of the protective shield 10 in either a clockwise or counterclockwise direction around the shaft of the penis and head of the penis such that the protective shield 10 engages the skin of the penis and then engages a predetermined portion of itself in a securable manner. It should be noted that the free end of the protective shield 10 may or may not be the edge having the remover mechanism 14 thereon. The protective shield 10 may be capable of being releasably securable to itself and the male penis to allow for daily use by a male. Therefore, after the protective shield 10 is wrapped around the head and predetermined portion of the shaft of the penis and a predetermined portion of itself the installation will be complete as shown in FIG. 3. The remover mechanism 14 may extend from the wrapped shield 10 a predetermined distance to allow for easy removal of the protective shield 10 after use. The protective shield 10 may stay attached to the penis during everyday use by the male and may allow for the head of the penis to be protected such as that afforded by the natural foreskin in uncircumcised men. When the user wants to remove the protective shield 10 from the penis, the user will grab the pull string 14 or the outer short edge of the body 12 and rotate the protective shield 10 in the direction opposite they applied the protective shield 10 to the penis. The simple rotation of the pull string 14 in the opposite direction of the application will allow for easy removal of the shield 10 from the penis for urination and sexual activity. It is also contemplated that the protective shield 10 may only cover the head of the penis or may cover the entire shaft of the penis along with the head of the penis. As shown in the figures, it is also contemplated that the protective shield 10 may extend a predetermined distance beyond the tip of the head of penis to ensure complete protection and coverage of the head of the penis from abrasion, wear and the like during use.

The present invention has been described in an illustrative manner. It is to be understood that the terminology, which has been used, is intended to be in the nature of words of description rather than of limitation.

Many modifications and variations of the present invention are possible in light of the above teachings. Therefore, within the scope of the appended claims, the present invention may be practiced otherwise than as specifically described.

What is claimed is:

1. A protective shield for a male penis, said shield comprising:
a rectangular shaped member, said member is flexible and removably securable to itself;
a remover mechanism is secured to an end of said member; and
a guideline arranged along at least one edge of said member.

2. The shield of claim 1 wherein said member is made of cellophane.

3. The shield of claim 1 wherein said member is approximately twelve inches by four and one half inches.

4. The shield of claim 1 wherein said guideline is arranged along a second edge of said member.

5. The shield of claim 4 wherein said second edge is one of a shorter edge or longer edge of said member.

6. The shield of claim 4 wherein said guideline extends continuously along said first edge and said second edge.

7. The shield of claim 4 wherein said guideline having a predetermined color.

8. The shield of claim 1 wherein said member is made of a plastic material.

9. The shield of claim 1 wherein said member is made of a polyethylene material.

10. The shield of claim 1 wherein said member having a predetermined color.

11. The shield of claim 1 wherein said remover mechanism is a string.

12. The shield of claim 11 wherein said string is secured to a shorter edge of said member.

13. The shield of claim 12 wherein said string extends a predetermined distance from said edge of said member.

14. A method of protecting a male penis, said method comprising the steps:
aligning a protective shield with a head and a predetermined portion of the shaft of the penis; and
wrapping said predetermined shield around said portion of said shaft and said head of the penis.

15. The method of claim 14 wherein said protective shield has a generally rectangular shape and is aligned along a longitudinal line of the penis.

16. The method of claim 14 wherein said protective shield is made of a flexible plastic material.

17. The method of claim 14 wherein said protective shield having a guideline arranged along at least one edge thereof.

18. The method of claim 14 wherein said protective shield having a remover mechanism secured to one end thereof.

19. The method of claim 14 wherein said step of wrapping has said shield removably engaged to itself at predetermined locations.

20. The method of claim 17 wherein said guideline is arranged along a second edge of said protective shield.
